(19) United States
(12) Patent Application Publication
(10) Pub. No.: US 2022/0061665 A1
(43) Pub. Date: Mar. 3, 2022

(54) System And Apparatus For Detecting Psychological State of a Signal Receiver

(71) Applicant: Fawzan Alqudairi, Sarasota, FL (US)

(72) Inventor: Fawzan Alqudairi, Sarasota, FL (US)

(21) Appl. No.: 17/305,089

(22) Filed: Jun. 30, 2021

(30) Foreign Application Priority Data
Aug. 30, 2020 (SA) GC 2020-40386

(51) Int. Cl. A61B 5/00; A61B 5/024

(52) U.S. Cl. CPC A61B 5/0004; A61B 5/02438; H04B 1/38; A61B 5/7221; A61B 5/681

(57) ABSTRACT

In summary, the present invention, in some embodiments thereof is predicated upon systems and methods for the detection of the psychological or physical state of a signal receiver. The system comprises of a signal transmitter coupled with a signal receiver, a signal receiver coupled to a signal transmitter, and a sensor element coupled to the body capable of determining the heart rate of the wearer. In a exemplary embodiment, a first transmitter transmits a request for the physiological state of the wearer of a sensor to a signal receiver. The signal receiver, preferably coupled to the sensor obtains the heart rate from the sensor, which measures and transmits back to the receiver a heart-rate, which in turn determines the physiological state of the wearer of the sensor to determine the degree of acceptance, satisfaction, rejection or anger.

### TECHNICAL FIELD

The present invention, in some embodiments thereof is predicated upon systems and methods for the detection of the psychological or physical state of a signal receiver wearer, and specifically to a system comprising a first transmitter coupled to a receiver, a receiver coupled to a transmitter and a sensor disposed on the body of a wearer.

### BACKGROUND OF THE INVENTION

The present teachings in some embodiments thereof are predicated upon systems and methods for the detection of the psychological or physical state of a signal receiver wearer. More specifically, the current invention relates to the field of transmitting and receiving signals, wherein the received signal contains a text message, an audio or video clip, a picture or similar stimulant, whether it is between two persons engaging directly in a mutual conversation or through a communication platform. The invention is applicable wherever a receiver and a transmitter are this signal.

In the present, signal transmitters are not adapted to receive the correct and accurate expression by a receiver of a signal stimulant. Typically, the recipient's expression is often manual and does not express the opinion of the signal receiver accurately. It is thus an objective of this invention to improve this signal process by the use of the system and apparatus that will be herewith disclosed in the descriptions of the current invention. The current invention would improve the attractiveness and flexibility of signal receivers to be able to accurately receive the response of a receiver according to the degree of comfort and conviction of the material displayed in the transmitted signal.

It is thus an object of the current invention to make it possible for any such party participating in the system anticipated by the current invention, to know the opinion or reaction of the receiver to this signal in a real and realistic way without forgery or courtesy. It may also be desirable for a receiver of a communication to know the truth nature of their feelings and their opinion, without external influences that sometimes compel them to express an insincere opinion. The current invention specifies the degree of satisfaction, acceptance or rejection, and expresses the opinion in a realistic manner, to aid in making decisions that will be taken based on the true state of the receiver of the signal.

In prior art implementations, the expressed opinion for a received signal may be merely courtesy and the opinion may change easily because it was not a real opinion from the beginning and this may affect decisions that will be taken based on the responses to these signals. However, in the current invention the expressed liking or dislike towards a signal received will be accurate and may further be adapted as a percentage dependent upon the measured heartbeat at the time of receiving the signal. Further, the percentage may change depending on the measured heartbeat, and can be deduced as a true reflection of what the receiver feels towards the received signal, and as a consequence help in making the right decision.

### SUMMARY OF THE INVENTION

The following summary is an explanation of some of the general inventive steps for the system, method, architecture and apparatus in the description. This summary is not an extensive overview of the invention and does not intend to limit the scope beyond what is described and claimed as a summary.

In summary, the present invention, in some embodiments thereof, relates to detection of the psychological or physical state of a signal receiver wearer, wherein it is provided a computer-implemented system and methods for the detection of the psychological or physical state of a signal receiver wearer, and specifically to a system comprising a first transmitter coupled to a receiver, a receiver coupled to a transmitter and a sensor disposed on the body of a wearer. In an exemplary embodiment, the current invention uses the heartbeat of the receiver, wherein a sensor is placed on the body of the wearer, and the output processed to determine the level of the heartbeat, and accordingly know their feeling of rejection, anger, or satisfaction and approval of the received signal and the degree of approval by a percentage, preferably taking into account any changes owing to a physical activity at the time of receiving the signal or in such cases where chronic diseases and medical history are involved. Preferably, chronic illnesses and physical activity information may further be perceived and processed by a sensor fixed around the wrist of the signal receiver.

### BRIEF DESCRIPTION OF FIGURES

The novel features believed to be characteristic of the illustrative embodiments are set forth in the appended claims. The illustrative embodiments, however, as well as a preferred mode of use, further objectives and descriptions thereof, will best be understood by reference to the following detailed description of one or more illustrative embodiments of the present disclosure when read in conjunction with the accompanying drawings, wherein:

FIG. 1 of the diagrams is an exemplary illustration of a system of the description of the current invention.

FIG. 2 of the diagrams is an exemplary illustration of the operation of a signal transmitting device.

FIG. 3 of the diagrams is an exemplary illustration of the operation of a signal receiving device.

FIG. 4 of the diagrams is an exemplary illustration of the operation of a heartrate sensing device disposed on a receiver's body.

### DETAILED DESCRIPTION

Hereinafter, the preferred embodiment of the present invention will be described in detail with reference to the accompanying drawings. The terminologies or words used in the description and the claims of the present invention should not be interpreted as being limited merely to their common and dictionary meanings. On the contrary, they should be interpreted based on the meanings and concepts of the invention in keeping with the scope of the invention based on the principle that the inventor(s) can appropriately define the terms in order to describe the invention in the best way.

It is to be understood that the form of the invention shown and described herein is to be taken as a preferred embodiment of the present invention, so it does not express fully the technical spirit and scope of this invention. Accordingly, it should be understood that various changes and modifications may be made to the invention without departing from the spirit and scope thereof.

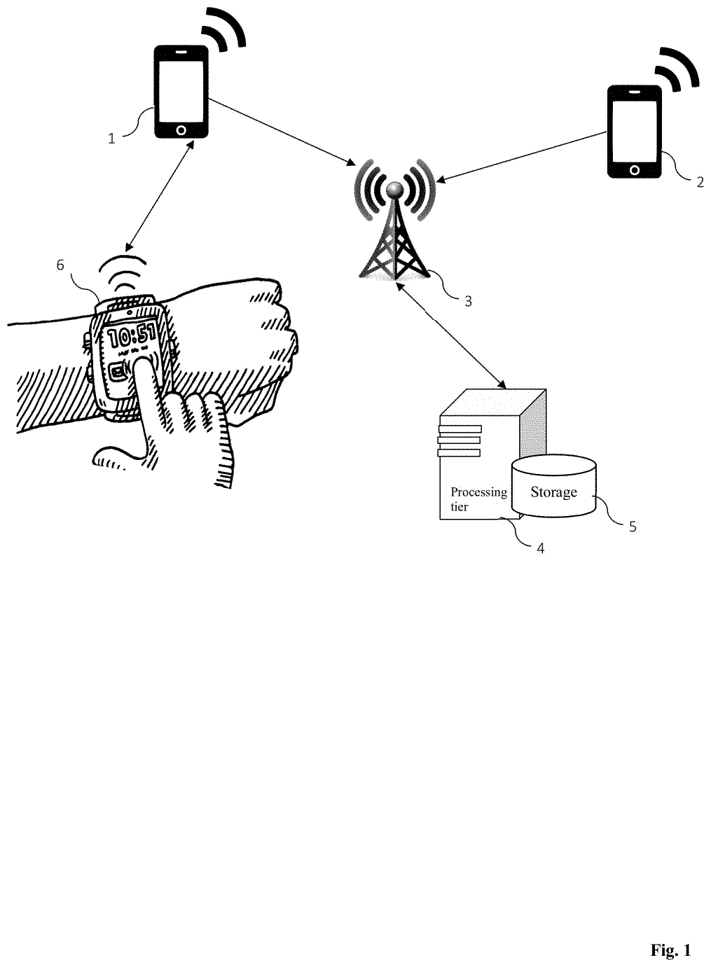

In a first exemplary embodiment according to FIG. 1 of the diagrams is an exemplary illustration of a system of the description of the current invention. In the diagram illustrated as 2, is a signal transmitting device, preferably comprising at least in part of signal generation mechanism, a signal transmitter, a signal receiver and a means to display a received signal. In some embodiment, the device 2 may embody a portable computing device. Preferably, the signal may comprise of a text message, an audio or video clip, a picture or similar stimulant, electromagnetic wave, mechanical or any such, which may be transmissible between any number of devices within a local area network such as with Bluetooth, Wi-Fi, NFC or a wide area network such as with the interne, mobile broadband, directly between devices or through an intermediary processing platform comprising at least in part of a processing tier 4 and a data storage 5.

Illustrated in the same FIG. 1, is a network 3, which may embody the internet, Wi-Fi, Bluetooth, local area network, wide area network, fibre optic, mobile broadband, 4G, 5G, 6G or any such implements that would enable the transmission of packets of data between any number of devices, and specifically in the context of the current invention, the transmission of signals between the devices participating in the implementation of the invention. The signal transmitting device 2, may suitably comprise of at least a memory, one or more processors, a network interface, a display apparatus and would be configured with a suitable computer program product comprising of a sequence of instructions on its memory, which when executed by said processor or processors, would make the device to transmit a signal to a receiver, receive the status of the receive at the time of transmission and display the psychological state of the receiver at the transmission and in reaction to the signal.

Further still in the same FIG. 1 is a device 1, which according to the current invention embodies a signal receiving device, preferably comprising at least in part of signal reception mechanism, a signal receiver and a signal transmitter. Preferably, the signal may comprise of a text message, an audio or video clip, a picture or similar stimulant, electromagnetic wave, mechanical or any such, which may be transmissible between any number of devices within a local area network such as with Bluetooth, Wi-Fi, NFC or a wide area network such as with the internet, mobile broadband, directly between devices or through an intermediary processing platform comprising at least in part of a processing tier 4 and a data storage 5.

The signal receiving device 1, may suitably comprise of at least a memory, one or more processors, a network interface, a display apparatus and would be configured with a suitable computer program product comprising of a sequence of instructions on its memory, which when executed by said processor or processors, would make the device to receive a signal from a transmitter, transmit the status of the receiver at the time of receiving the signal and transmit the psychological state of the receiver at the reception of and in reaction to the signal. Further still in the same FIG. 1, is a sensor 6, which according to the current invention may embody a smart-watch capable of measuring the heart rate of the wearer. Preferably, the signal receiver's level of heartbeat in normal conditions is saved in the sensor or a receiver device 1 coupled to said sensor and as such, it is can be established through a suitable device configuration, such as using a computer program product comprising of a sequence of instructions configured on the device memory, when the state of anger or dissatisfaction the level of the heartbeat rises and thus expresses non satisfaction with the received signal. The improved technical utility that characterizes the disclosed approach would make its possible application to a large segment of users, so it can be said that one general objective of this invention is to find a method for determining the opinion about the received signal more accurately, automatically, new and its industrial application provides a better utility than the prior art manual methods.

The disclosed system and methodology is an obvious improvement to the currently recognized manual methods, which include a configuration for either the like or dislike of a received signal, and which cannot be guaranteed as accurate because the reporting may be wrong or not express the true opinion of the receiver. It is noteworthy that prior art descriptions do not provide for any accurate measurement of liking or disliking, which provides the user with limited options to express an opinion sometimes lacks credibility or transparency.

In some embodiments, a processing tier 4 comprising at least in part of a memory, a processor and a storage device 5, may be provided as an intermediary medium for the transmission of signals between the devices 1 and 2. It may further be adapted for the accessible storage of a signal, which forms part of the object of transmission including but not limited to a text message, an audio or video clip, a picture or similar stimulant, electromagnetic wave, mechanical or any such, which may be transmissible between any number of devices within a network of any type.

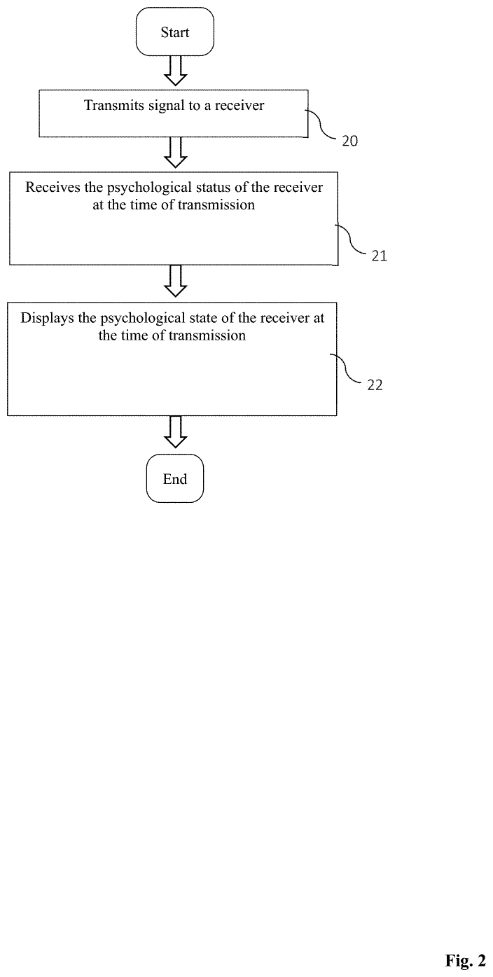

In an exemplary embodiment as provided in the FIG. 2 of the diagrams is an exemplary illustration of the operation of a signal transmitting device. A suitably configured signal transmitting device transmits a signal to a receiver device in a network, local or wide area as in the step 20. In the step 21, the signal transmitting device may receive the psychological state of the receiver as at the time of transmission of the signal. This psychological state of the receiver may be received after the determination of the level of the heartbeat of the receiver by a corresponding recipient device, and accordingly know their feeling of rejection, anger, or satisfaction and approval of the received signal and the degree of approval by a percentage, preferably taking into account any changes owing to a physical activity at the time of receiving the signal or in such cases where chronic diseases and medical history are involved. In the step 22, the method entails a further display of the psychological state of the receiver on a suitable display apparatus of the signal transmitting device.

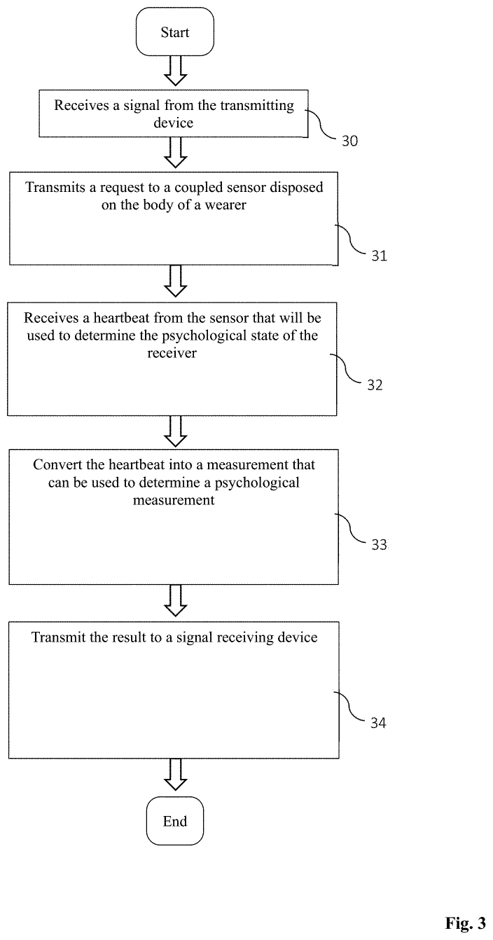

According to an embodiment according FIG. 3 of the diagrams is an exemplary illustration of the operation of a signal receiving device. A suitably configured signal receiving device such as 1, in the FIG. 1, receives a signal from a suitably configured signal transmitting device such as 2, in the FIG. 1 in a step 30. The signal receiving device subsequently in a step 31, transmits a request to a coupled sensor disposed on the body of a receiver for a measured heart-rate. Next, in the step 32, the signal receiving device receives the heart-rate from the coupled sensor, as illustrated in the FIG. 1 above, wherein the received heart-rate may be used to determine the psychological state of the receiver. Subsequently, in the step 33, the received heart-rate or heartbeat measurement is converted into a psychological state of the receiver of rejection, anger, or satisfaction and approval of the received signal and the degree of approval by a percentage, preferably taking into account any changes owing to a physical activity at the time of receiving the signal, or in any such cases where chronic diseases and medical history are involved. Finally, in the step 34, there is a transmission of the determined psychological state to a signal transmitting device. Preferably, the transmission is performed via any such network implementation.

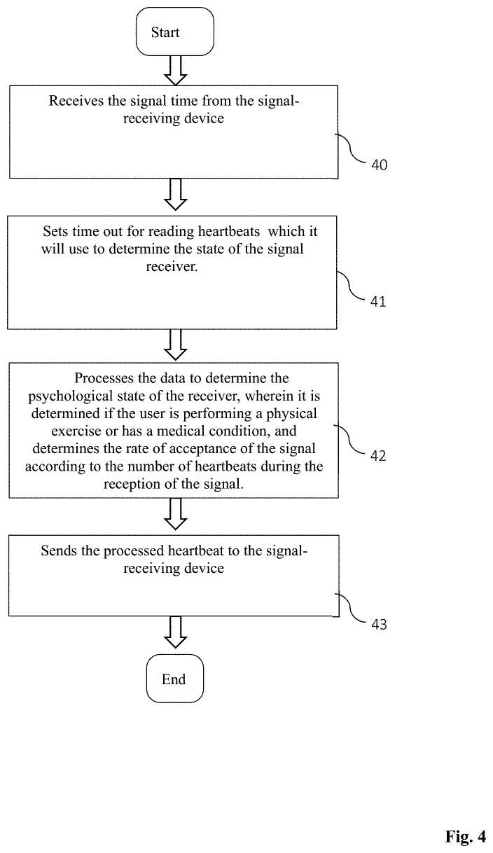

In a further and final exemplary embodiment according to the FIG. 4 of the diagrams is an illustration of the operation of a heart-rate sensing device or sensor disposed on a receiver's body. Beginning at the step 40 of the operation is receiving a request at the time of a signal transmission for the measured heart-rate. Subsequently, in the step 41, the sensor or heart-rate sensing device disposed on or inside a receiver's body performs an action of reading of the heart-rate that will be used to determine the receiver's psychological state, the device may further set a time-out for the reading/measurement of the receiver's heart-rate. Subsequently, in the step 42, the device processes the heart-rate measurement data to determine the psychological state of the receiver, wherein it is determined if the user is performing a physical exercise or has a medical condition, and determines the rate of acceptance of the signal according to the number of heartbeats during the reception of the signal. Finally, in 43, the processed heartbeat measurement is transmitted to the signal-receiving device.

In order to do improve the prior art implementations, the current invention provides a distinctive method for signal-receiving and transmitting devices to be used more effectively through this signal-receiving sensor , which is disposed in receiving devices. Further, the method disclosed for the current invention's receiving sensor is new, inexpensive and generally very accessible to the public to provides the best solution for determining accuracy of responses at the signal receiver.

According to an exemplary embodiment, the signal transmitting device comprises a signal receiver, capable of receiving the psychological state measured an determined at the receiver.

According to an exemplary embodiment, the signal receiver device comprises a signal transmitter, capable of transmitting the psychological state measured.

According to an exemplary embodiment, the heart-rate sensing device or sensor, may be configured capable of measuring the heart-rate, while taking into account any variations within a specified period, such as 24 hours, from which the minimum number of heart beats per minute and the maximum number of heart beats per minute may be determined, and which would help deduce the normal heartbeats level of the signal receiver and as such enable the exclusion of variations caused by physical exercise at the time of receiving a signal, and further exclude variations caused by a medical condition.

According to an exemplary embodiment the heart-rate sensor will comprise of a measurement history of the signal receiver to determine variations caused by physical activity, such as sport, at the time of receiving the signal, and as such aid in the processing of measured data to determine the correct measurement of the heartbeat and transmits it to the signal- receiving device.

According to an exemplary embodiment, the receiver of the heart-rate measurements may be adapted to process the heart beat or heart-rate measurement in such manner that the correct reading is determinable when receiving the signal, by factoring interruptions during measurement and historical measurements, wherein the correct psychological or state of the signal receiver may be determined more accurately.

The foregoing is provided for purposes of illustrating, explaining, and describing embodiments of the present invention. Modifications and adaptations to these embodiments will be apparent to those skilled in the art and may be made without departing from the scope or spirit of the invention.

### INDUSTRIAL APPLICATION

The current invention technology is applicable to signal receivers, and more specifically, the method of applying to industry relates to the detection of the psychological state of a receiver of a signal to improve the overall accuracy of systems that detect the psychological state of a receiver of a signal.

What is claimed is:

1. A system for detecting the psychological state of a signal receiver, the system comprising of:
a signal transmitting device comprising of a signal transmitter, and signal receiver, configured capable of:
transmitting a signal to a receiver;
receiving from a signal receiving device the psychological status of the receiver at the time of transmission of the signal;
displaying the received psychological state of the receiver at the time of transmission;
a signal receiving device configured capable of:
receiving a signal from a transmitting device;
transmitting a request for a measured heartbeat rate to a coupled sensor on the body of a wearer;
receiving a heartbeat from a suitably configured sensor to be used to determine the psychological state of the receiver;
converting the heartbeat measurement received from the sensor into a measurement that can be used to determine a psychological measurement, and;
transmitting the determined psychological measurement to a signal transmitting device, and;
a heart-rate measuring sensor configured capable of:
receiving from a signal receiving device a request at the time of a signal transmission for the measured heart-rate;
performing an action of measuring the heart-rate that will be used to determine the receiver's psychological state;
processing the heart-rate measurement data to determine the psychological state of the receiver, wherein it is determined if the user is performing a physical exercise or has a medical condition to obtain an accurate heart-rate measurement, and;
transmitting the processed heartbeat measurement to the signal receiving device.

2. The system as in claim 1, wherein the heart-rate measuring sensor is a smart-watch.

3. The system as in claim 1, wherein the heart-rate measuring sensor is a cardiac monitor.

4. The system as in claim 1, wherein the heart-rate measuring sensor is further configured capable of measuring the heart-rate, while taking into account any variations within a specified period, such as but not limited to 24 hours, from which the minimum number of heart beats per minute and the maximum number of heart beats per minute may be determined.

5. The system as in claim 1, wherein the heart-rate measuring sensor is further configured capable to comprise of a measurement history of the signal receiver to determine variations caused by physical activity or medical condition.

6. The system as in claim 1, wherein the signal receiving device adapted to process the heart beat or heart-rate measurement in such manner that the correct reading is determinable when receiving the signal, by factoring interruptions during measurement and historical measurements, wherein the correct psychological or state of the signal receiver may be determined more accurately

7. The system as in claim 1, wherein the signal receiving device comprises of a signal receiver and a signal transmitter.

8. The system as in claim 1, wherein the signal transmitting device comprises of a signal receiver and a signal transmitter.

9. The system as in claim 1, wherein the signal receiving device is configured capable of coupling to a heart-rate measuring sensor.

10. A method of detecting the psychological state of a signal receiver, the method comprising of:
transmitting by a signal transmitting device a signal to a signal receiver device;
receiving by a signal transmitting device from a signal receiving device the psychological status of the receiver at the time of transmission of the signal, and;
displaying by a signal transmitting device the received psychological state of the receiver at the time of transmission.

11. The method of claim 10 further comprising of:
receiving by a signal receiving device a signal from a signal transmitting device;
transmitting by a signal receiving device a request for a measured heartbeat rate to a coupled sensor on the body of a wearer;
receiving by a signal receiving device a heartbeat from a suitably configured sensor to be used to determine the psychological state of the receiver;
converting by a signal receiving device the heartbeat measurement received from the sensor into a measurement that can be used to determine a psychological measurement, and;
transmitting by signal receiving device the determined psychological measurement to a signal transmitting device.

12. The method of claim 10 further comprising of:
receiving by a heart-rate measuring sensor from a signal receiving device a request at the time of a signal transmission for the measured heart-rate;
performing by a heart-rate measuring sensor an action of measuring the heart-rate that will be used to determine the receiver's psychological state;
processing by a heart-rate measuring sensor the heart-rate measurement data to determine the psychological state of the receiver, wherein it is determined if the user is performing a physical exercise or has a medical condition to obtain an accurate heart-rate measurement, and;
transmitting by a heart-rate measuring sensor the processed heartbeat measurement to the signal receiving device.

13. The method of claim 10 further, wherein the signal comprises of a text message.

14. The method of claim 10 further, wherein the signal comprises of an audio file.

15. The method of claim 10 further, wherein the signal comprises of video file.

16. The method of claim 10 further, wherein the signal comprises of a picture.

17. The method of claim 10 further, wherein the signal comprises of an electromagnetic wave.
